(19) United States
(12) Patent Application Publication
(10) Pub. No.: US 2007/0185427 A1
(43) Pub. Date: Aug. 9, 2007

(54) Adhesive bandage with reduced pain during removal

(76) Inventor: Chung-Yu Lin, Kaohsiung City (TW)

Correspondence Address: BACON & THOMAS, PLLC, 625 SLATERS LANE, FOURTH FLOOR, ALEXANDRIA, VA, 22314, US

(21) Appl. No.: 11/652,630

(22) Filed: Jan. 12, 2007

Related U.S. Application Data
(60) Provisional application No. 60/758,544, filed on Jan. 13, 2006.

(51) Int. Cl. A61F 15/00

(52) U.S. Cl. 602/57

(57) ABSTRACT

The present invention relates to an adhesive bandage with reduced pain during removal, whose flat strip substrate has a sticking front, a non-sticking back and four sides. And, both areas of said sticking front and non-sticking back are greater than those of four sides; The feature is: a strip of non-glued area is disposed between both of opposite parallel lengthwise lateral sides and two strips of glued area are parallel juxtaposed on both lengthwise sides of said non-glued area respectively on said sticking front, wherein a pair of glue-less indentations are formed on both end sides of said non-glued area but not adjacent said glued area of said strip substrate and being flush with both crosswise end sides; When the sticking front of the strip substrate sticks and presses on the skin epidermis around the wound, the middle non-glued area does not contact and stick on the skin epidermis S; Hence, when said adhesive bandage is to be ripped off upon changing medical dressing, our fingers can take anyone glue-less indentation on the strip substrate as grip fulcrum for lifting up to achieve the purpose of tearing off said strip substrate from the skin epidermis with effect of no painful feeling by such tearing off action.

### FIELD OF THE PRESENT INVENTION

The present invention relates to the common household nursing and medical treatment adhesive bandage and sticking plaster also including the sticking strip of snore-preventing usage and various adhesive tapes of packaging or sealing products in people's livelihood and industry, particularly stresses the multiple enhanced effects of simple rapid in ripping operation, reducing production manufacturing cost, promoting the environment protection responsibility, and eliminating the skin painful side effect, thereby increasing the total integral business competitive force.

### BACKGROUND OF THE INVENTION

It is universally known that all different kinds of adhesive bandage and sticking plaster have closed relationship with our livelihood, ranging from the daily light wound of chafe or cut and the slight symptom of different sore or ache to the variety of serious trauma in surgery, all these need fully rely on the various suitable adhesive bandage and sticking plaster as accidental and incidental treatment means; Moreover, the package of livelihood articles or industrial products is also the use of adhesive tape; For sealing or wrapping need in the daily live, the adhesive tape is used too.

The kind of all the adhesive tapes used in different fields aforesaid is quite a few; For the various adhesive bandages of nursing medical grade, they are amply disclosed in some websites such as www.webhospital.org.tw, www.3m.com, www.allfit.com.cn etc.; However, the serious drawback for the adhesive bandage used in the epidermis of human skin is difficult to tear off after a period of sticking on the skin; namely, a tiny edge or corner of adhesive bandage is first lifted by one finger, then tear off the whole adhesive bandage from the skin by two fingers. Therefore, how to achieve the preceding task of lifting such tiny edge or corner of adhesive bandage becomes very important and necessary. Owing to the conventional nursing and medical treatment adhesive bandage does not have any contrivance of easily lifting such tiny edge or corner of adhesive bandage, the medical doctor or nurse is forced to tackle such task by his or her own way. For observation by the inventor of the present invention from practical training as intern to becoming a qualified doctor in all hospitals serviced, the most common way adopted by those other medical doctors or nurses is to lift the edge or corner of the adhesive bandage by a blunt flat object such as ball-point pen. If it happens to have no suitable blunt flat object on the spot, the single finger is obliged to be used for picking and lifting such tiny edge or corner of adhesive bandage up to many times occasionally, which suffers the patient from pain on the skin even to forbear from speaking. Thus, that not only extends the time in changing medical dressing but also increases the frequency of extra torture on the patient. If said finger picking or lifting way in the change of nursing and medical treatment adhesive bandage is applied in the household for the daily light wound of chafe or cut, it only causes extra pain on the skin mentioned above without any harmful effect. However, for mass patients in the hospital under the requirement of timing effect and aseptic technology task, said finger picking or lifting way might prolong the curing time for the injured patient or increase the possibility of infection. Studying and probing the cause, there is no “convenient ripping contrivance” on the existing conventional nursing and medical treatment adhesive bandage, unfortunately, no better solution being worked out up to now.

Besides, the sticking strip of snore-preventing as disclosed in the U.S. Pat. No. 6,089,232 and US patent application published No.US2004/0089310 is shown in the FIGS. 1 through 5. The sticking front of the sticking strip 10 is all spread over glue or gum G so as to stick it on the mouth area covering upper lip L1 and lower lip L2 (as shown in the FIGS. 1, 2, and 4 as well as view-a of the FIG. 4). For people with poor dermal adaptability might be involved in the allergic or un-adaptable risk owing to upper lip L1 and lower lip L2 being more fragile and sensitive than skin around the mouth. Moreover, the procedure of tearing off said sticking strip of snore-preventing after sleep, the drawback of being not easy in tear-off operation aforesaid and the painful feeling by pulling the glued sticking strip away upper lip L1 and lower lip L2 will still happen (as shown in the FIG. 3 and view-c of the FIG. 3 as well as the FIG. 5 and view-c, view-d of the FIG. 5).The inadequate and incomprehensive design is the main reason why said sticking strip of snore-preventing is not in a great quantity promoted to use yet.

For the sealing adhesive tape used in the livelihood and industrial goods, disclosed in the Taiwan utility patent publication No.485963, there is a tape ripping structure as shown in the FIG. 6. The main element of rectangular flakes 21 made of non-adhesive anti-break material are disposed on the adhesive back 22 of the adhesive tape in the manner of adequate dividing pitch P such that said tape ripping structure having separating arrangement of multiple flakes 21 after winding said adhesive tape 20 into roll product (as shown in the FIG. 7). When the adhesive tape needs to be ripped off from the sticking sealed box body 1, the operation is shown as in view-A of the FIG. 8. First, from the bottom side of any flake 21 pick the flake 21 up away the box body 1 by finger (as shown in view-B of the FIG. 8), then tear off said adhesive tape 20 to achieve the purpose of removing sealant need not using any knife for cutting. Form above description, it is known that although said tape ripping structure is helpful to improve the operation of ripping off the adhesive tape 20, some drawbacks of imperfect still exist as depicted below:

1. As shown in view-A of the FIG. 8, the end vicinity of the adhesive tape 20 must remain a flake 21 for ripping off when apply it to seal the box body 1 so as to achieve the convenience in future tearing off operation; Otherwise the cutting tool is drawn for help as same as it happens in the conventional adhesive tape. For consuming user, the beforehand re-education in usage is necessary to make it practicable instead of enjoy the improvement of simplicity contrarily; thereby, that becomes the reason in making said product be unable sold for use in the market.

2. For all existing adhesive tape of mass production, the roll-type is always the end product for subsequent process of package and transportation load with preset specifications and cost control; said flake 21 affixed with the adhesive tape 20 being enrolled into roll-type end product will make its volume and total weight greater than those of conventional adhesive tape. Thus, said adhesive tape is not only unsuitable for existing post-process of package and transportation load but also increase the extra cost to weaken the business competitive force.

3. For production process, the whole automated production equipment and facility must be readjusted for extra materials in association with said flake 21 such that total material and manufacturing cost being increased accordingly. For the adhesive tape mass used in the livelihood and industry, not only the total accumulated cost will be immensely increased, but also an article is created in the recycling of environmental protection. In reality, that means the harm is first suffered before the benefit to be met.

In view of all the drawbacks described above in various field of adhesive tape usage, the inventor of the present invent actively studies and researches to develop and contrive out a better adhesive tape and adhesive bandage so as to eliminate all the drawbacks aforesaid.

### SUMMARY OF THE INVENTION

The present invention originates a flat adhesive bandage with reduced pain during removal, whose flat strip substrate has a non-glued area being disposed on the sticking front and a pair of glue-less indentations being formed on both end sides of said non-glued area; By means of said glue-less indentation, our fingers is provided a start grip fulcrum for lifting up to achieve the purpose of quickly tearing off said adhesive bandage from the skin epidermis with speeding up the completely ripping off effect in addition to the simplicity and easiness in ripping off said adhesive bandage; That is main object of the present invention.

The second object of the present invention is to provide a flat adhesive bandage with reduced pain during removal. Owing to the non-glued area on the sticking front of the strip substrate, the contact area of the sticking front in closely sticking with human skin epidermis will be decreased; Thereby, not only the painful feeling of tearing off action can be eliminated, but also the allergic risk caused by the skin contact can be reduced.

Another object of the present invention is to provide a flat adhesive bandage with reduced pain during removal. Owing to the non-glued area on the sticking front of the strip substrate, when it sticks and presses on the skin epidermis around the wound, the middle non-glued area does not contact and stick on the skin epidermis so that not only the contact area on the skin epidermis can be reduced but also any stimulus on the flesh bud of heal over place can be saved; Thereby, neither the drawback of contact infection on the medical dressing nor the drawback of stimulus and contamination of wound or flesh bud tissue by the glue constituent will happen so that it is conducive to the benefit in expediting heal over of the wound.

The further object of the present invention is to provide a flat adhesive bandage with reduced pain during removal, which can be applied in the sticking bandage of snore-preventing. By means of one strip said non-glued area and two strips said glued area on the sticking front of the bandage substrate, the sticking bandage can closely stick on the outer skin epidermis around the upper lip and the lower lip so that closed upper lip and lower lip is accommodated in said non-glued area without uncomfortable feeling caused by the sticking and stimulus from glue. Besides, it has both effects in simplicity and easiness in quickly ripping off said adhesive bandage in addition to elimination of painful feeling in tearing off.

The furthermore object of the present invention is to provide a flat adhesive bandage with reduced pain during removal, which is also applied in the sticking bandage of snore-preventing. Wherein the bandage substrate is further disposed into translucent or transparent flat shape and the pair strips of glued area on the sticking front of said bandage substrate are spread with color glue so that the ripping off efficiency and the management effect of induce arrangement can be increased by the variety and identification of the glue color.

Employing the contrivance of glued area and non-glued area, the present invention further originates a simple and easy tearing-off adhesive tape used in the article. The flat strip substrate has a sticking front, a non-sticking back and four sides. Wherein the areas of said sticking front and non-sticking back are greater than those of four sides; The feature is: a strip of non-glued area is disposed in the center of the sticking front and two strips of glued area are parallel juxtaposed on both lengthwise sides of said non-glued area respectively on said sticking front, wherein a pair of glue-less indentations are formed on both end sides of said non-glued area but not adjacent said glued area of said strip substrate and being flush with both crosswise end sides; when the strip substrate is applied to close stick and seal the article or in package, the operation way is same as that of conventional adhesive tape. If the adhesive tape is to be ripped off, just align the fingers with either glue-less indentation of both crosswise short end sides on the strip substrate as grip fulcrum for lifting up to achieve the purpose of tearing off whole said adhesive tape or unpacking.

Additionally, the present invention further provides a simple and easy tearing-off adhesive tape used in the article wherein an additional pair strips of parallel juxtaposed slimmer non-glued area are inwards disposed abut against said pair of parallel lengthwise long lateral sides of said strip substrate and adjacent to the pair strips of glued area on the sticking front of the strip substrate so that said pair of parallel lengthwise long lateral sides will not become dirty due to touching and accumulating dust after the strip substrate being enrolled into roll-type.

Moreover, the present invention further provides a simple and easy tearing-off adhesive tape used in the article. Owing to the non-glued area being disposed on the strip substrate with less glue spread over, the total weight of roll-typed adhesive tape end product is substantially decreased compare to conventional adhesive tape; Thus, it has better business competitive force in consequence of more labor-saving to carry and cost-saving in transportation.

Furthermore, the present invention further provides a simple and easy tearing-off adhesive tape used in the article. Owing to the non-glued area being disposed on the strip substrate with less glue spread over, not only the total quantity of glue consummation is accumulatively saved, but also the total energy consummation in enrolling process is decreased. Thus, both saving benefit in environment protection recycling and energy are created.

### BRIEF DESCRIPTION OF THE DRAWINGS

FIG. 1 is the first illustrative view of the conventional sticking strip of snore-preventing in manner of closely sticking on the mouth.

FIG. 2 is the second illustrative view of the conventional sticking strip of snore-preventing in manner of closely sticking on the mouth.

FIG. 3 is the operational illustrative view for the conventional sticking strip of snore-preventing in manner of tearing-off operation.

FIG. 4 is the third illustrative view of the conventional sticking strip of snore-preventing in manner of closely sticking on the mouth.

FIG. 5 is the operational illustrative view for the conventional sticking strip of snore-preventing of FIG. 4 in manner of tearing-off operation.

FIG. 6 is the perspective illustrative view for the conventional sealing adhesive tape used in livelihood and industry.

FIG. 7 is the planar illustrative view for the conventional sealing adhesive tape used in livelihood and industry.

FIG. 8-A is the first perspective operational illustrative view for the conventional sealing adhesive tape used in livelihood and industry.

FIG. 8-B is the second perspective operational illustrative view for the conventional sealing adhesive tape used in livelihood and industry.

FIG. 9 is the perspective illustrative view for the first exemplary embodiment of the present invention.

FIG. 10 is the application illustrative view for the first exemplary embodiment of the present invention.

FIG. 11 is the section illustrative view for the FIG. 10 taken along the line of c-c.

FIG. 12 is the application illustrative view for the conventional nursing and medical adhesive bandage.

FIG. 13 is the section illustrative view for the FIG. 12 taken along the line of d-d.

FIG. 14 is the illustrative view for the first exemplary embodiment of the present invention in manner of closely sticking on the elbow.

FIG. 15 is the illustrative view for the conventional nursing and medical adhesive bandage in manner of closely sticking on the elbow.

FIG. 16 is the illustrative view for the first exemplary embodiment of the present invention in manner of closely sticking on the knee.

FIG. 17 is the illustrative view for the conventional nursing and medical adhesive bandage in manner of closely sticking on the knee.

FIG. 18 is the operational illustrative view for the first exemplary embodiment of the present invention in manner of tearing-off operation from the elbow.

FIG. 19 is the operational illustrative view for the conventional nursing and medical adhesive bandage in manner of tearing-off operation from the elbow.

FIG. 20 is the operational illustrative view for the first exemplary embodiment of the present invention in manner of tearing-off operation from the knee.

FIG. 21 is the operational illustrative view for the conventional nursing and medical adhesive bandage in manner of tearing-off operation from the knee.

FIG. 22 is the perspective illustrative view for the strip substrate of the present invention in manner of winding into roll-type.

FIG. 23 is the perspective illustrative view for the second exemplary embodiment of the present invention.

FIG. 24 is the perspective illustrative view for the third exemplary embodiment of the present invention.

FIG. 25 is the perspective illustrative view for the forth exemplary embodiment of the present invention.

FIG. 26 is the perspective illustrative view for the fifth exemplary embodiment of the present invention.

FIG. 27 is the first illustrative view for the exemplary embodiment of the present invention applied in the large-sized sticking plaster.

FIG. 28 is the second illustrative view for the exemplary embodiment of the present invention applied in the large-sized sticking plaster.

FIG. 29 is the perspective illustrative view for the exemplary embodiment of the present invention applied in the sticking bandage of snore-preventing.

FIG. 30 is the first illustrative view for the present invention applied in the sticking bandage of snore-preventing in manner of closely sticking on the mouth.

FIG. 31 is the second illustrative view for the present invention applied in the sticking bandage of snore-preventing in manner of closely sticking on the mouth.

FIG. 32 is the third illustrative view for the present invention applied in the sticking bandage of snore-preventing in manner of closely sticking on the mouth.

FIG. 33 is the first illustrative view for the present invention applied in the sticking bandage of snore-preventing in manner of tearing-off operation.

FIG. 34 is the second illustrative view for the present invention applied in the sticking bandage of snore-preventing in manner of tearing-off operation.

FIG. 35 is the perspective illustrative view for the present invention applied in the article use in manner of simple and easy tearing-off adhesive tape operation.

FIG. 36 is the first perspective operational illustrative view for the present invention applied in the simple and easy tearing-off adhesive tape used in the article.

FIG. 37 is the second perspective operational illustrative view for the present invention applied in the simple and easy tearing-off adhesive tape used in the article.

FIG. 38 is the perspective illustrative view for the second exemplary embodiment of the present invention applied in the simple and easy tearing-off adhesive tape used in the article.

FIG. 39 is the side illustrative view for the second exemplary embodiment of the present invention applied in the simple and easy tearing-off adhesive tape used in the article.

FIG. 40 is the perspective illustrative view for the third exemplary embodiment of the present invention applied in the simple and easy tearing-off adhesive tape used in the article.

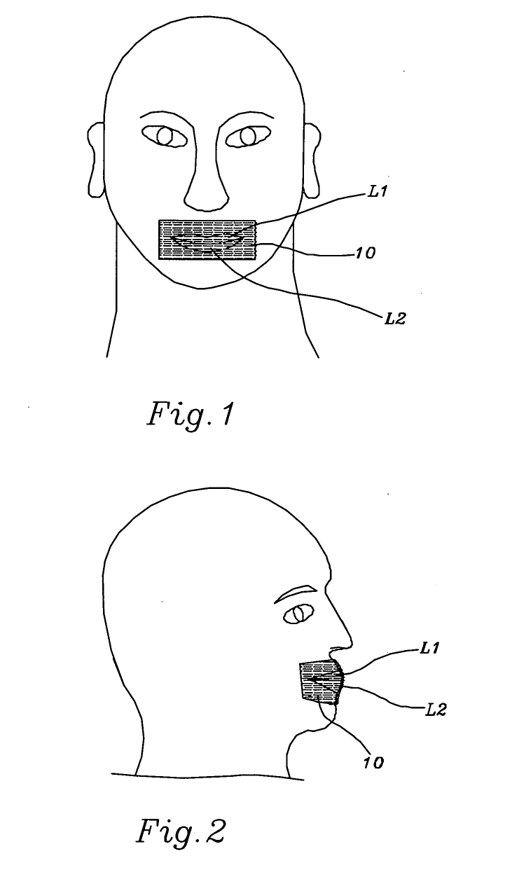

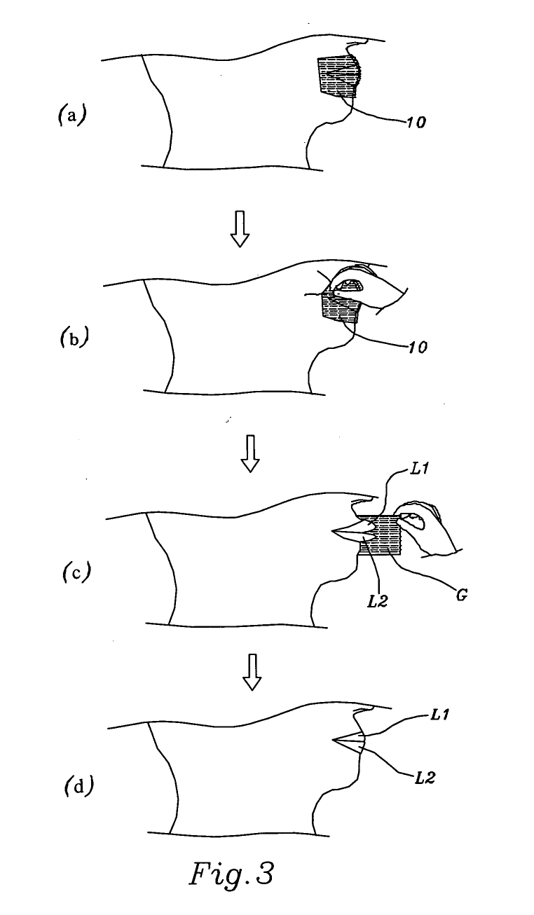

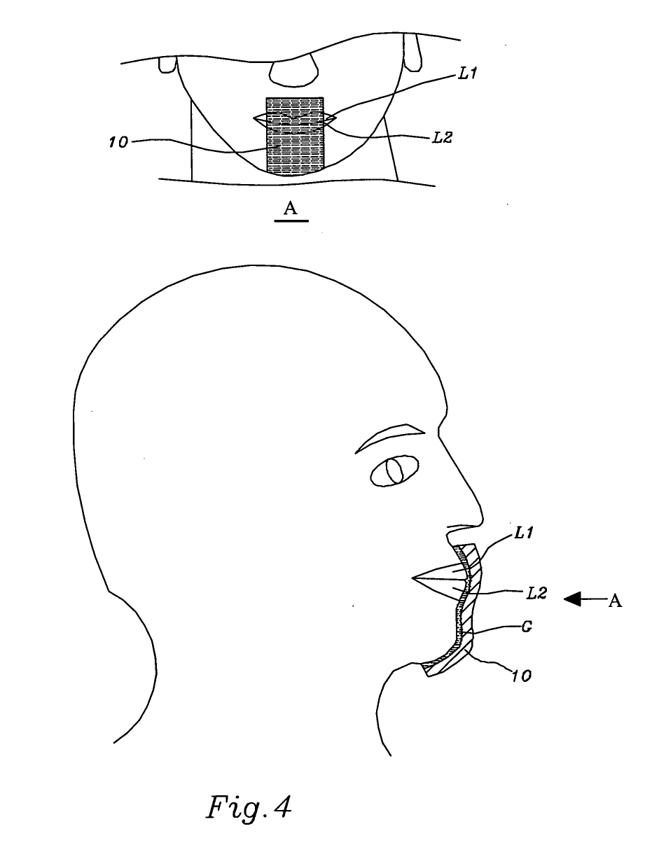

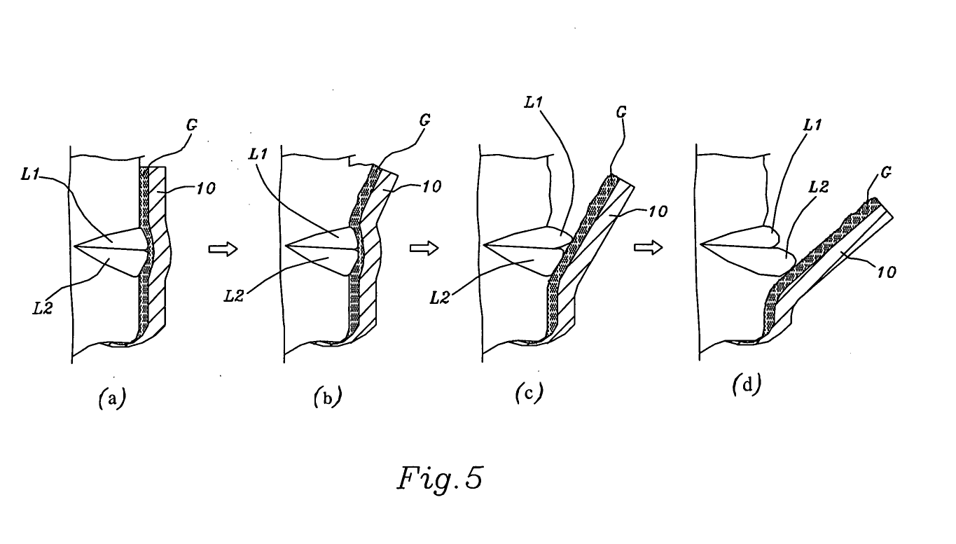

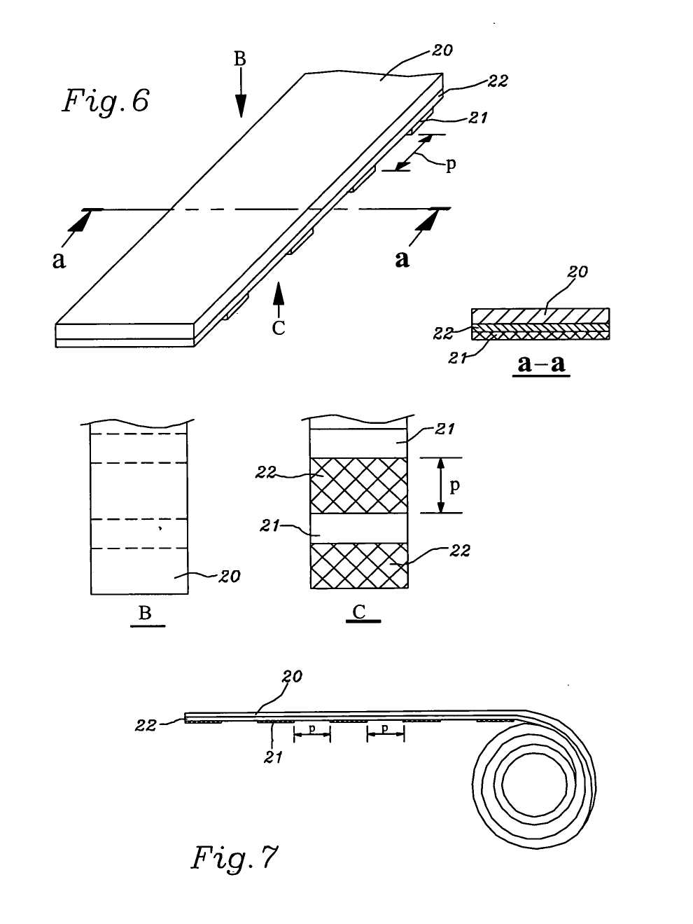

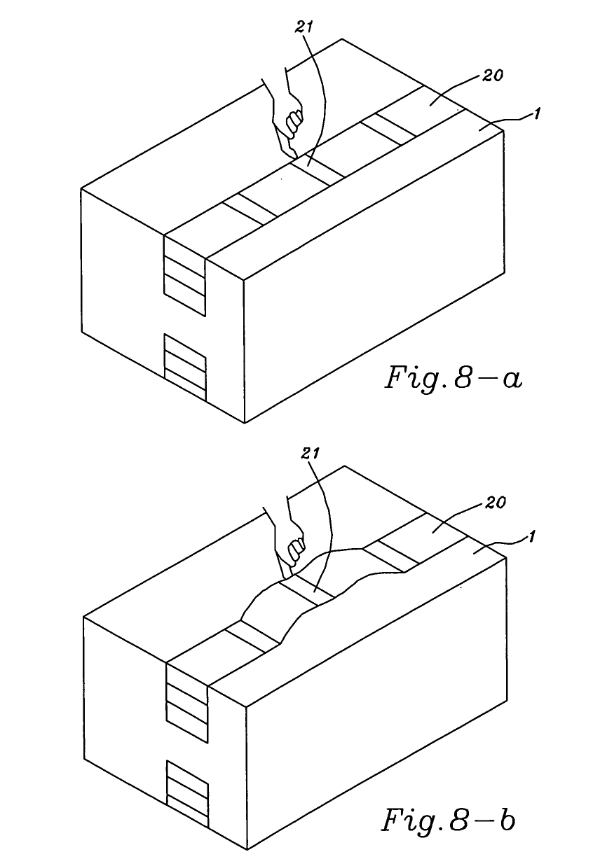

### DETAILED DESCRIPTION OF THE PREFERRED EMBODIMENTS

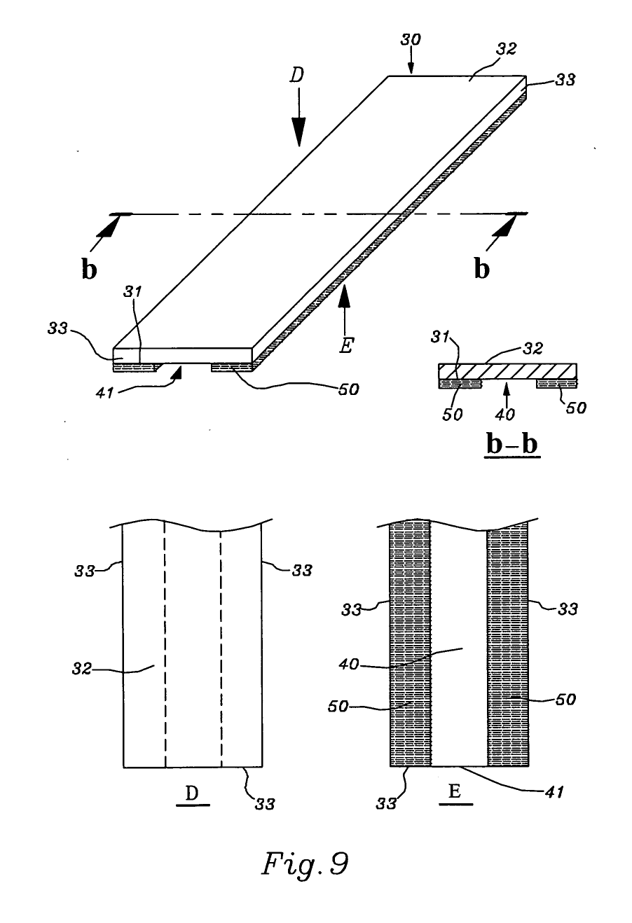

Referring to FIG. 9, an adhesive bandage with reduced pain during removal according to a first preferred embodiment of the present invention, which is a flat strip substrate 30 comprises a sticking front 31, a non-sticking back 32 and four lateral sides 33. The areas of said sticking front 31 and non-sticking back 32 are greater than the four lateral sides 33, wherein a strip of non-glued area 40 is disposed between both of opposite lateral sides 33 and two strips of glued area 50 are juxtaposed on both sides of said non-glued area 40 respectively on said sticking front 31. Therefore, a pair of glue-less indentations 41 are formed on both end sides of said non-glued area 40 but not adjacent said glued area 50 of said strip substrate 30 and being flush with both lateral sides 33. Besides, the strip substrate 30 can be either medical cosmetology adhesive tape or surgical adhesive tape in accordance with the application requirement.

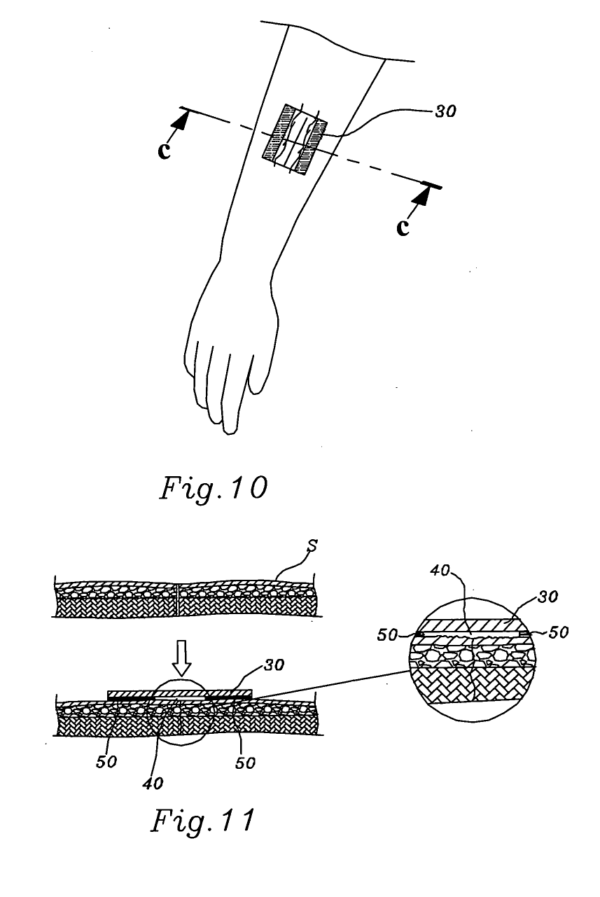

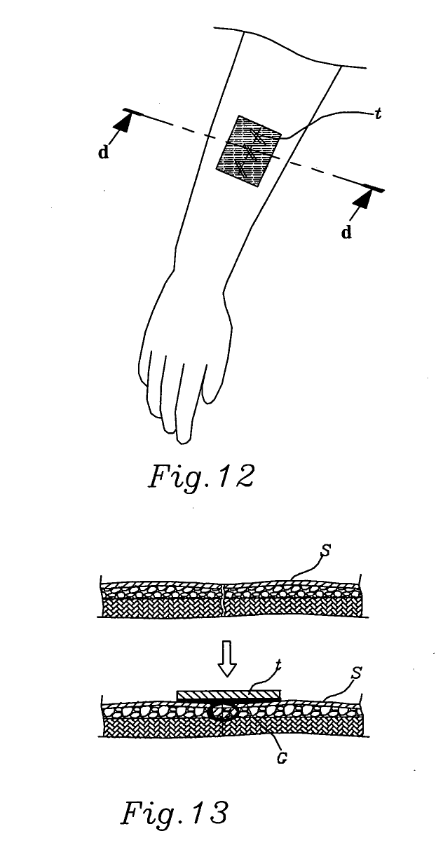

Please refer to the FIGS. 10 through 13, when the sticking front 31 of the strip substrate 30 of the present invention sticks and presses on the skin epidermis S around the wound, the middle non-glued area 40 does not contact and stick on the skin epidermis S as shown in the FIGS. 10 and 11 so that not only the contact area on the skin epidermis S can be reduced but also any stimulus on the flesh bud of heal over place can be saved; Thereby, neither the drawback of contact infection on the medical dressing nor the drawback of stimulus and contamination of wound or flesh bud tissue by the glue constituent will happen so that it is conducive to the benefit in expediting heal over of the wound. Contrarily, the sticking front by spreading the glue all over of the conventional medical adhesive bandage t will completely contact and stick on the wound of the skin epidermis S as shown in the FIGS. 12 and 13; Thereby, not only the stimulus on the flesh bud of heal over place but also the contact infection on the medical dressing will happen so that the heal over time of the wound will prolong.

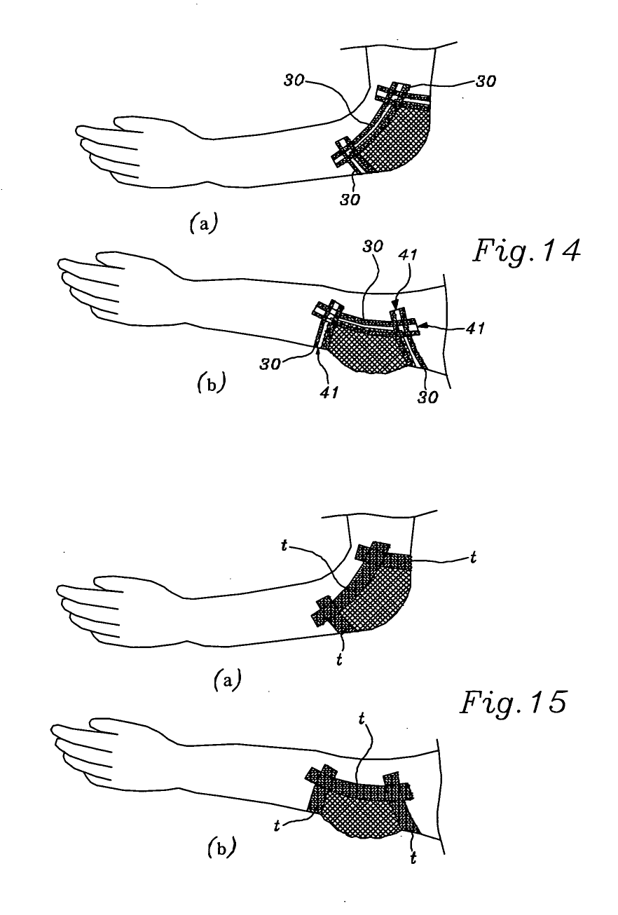

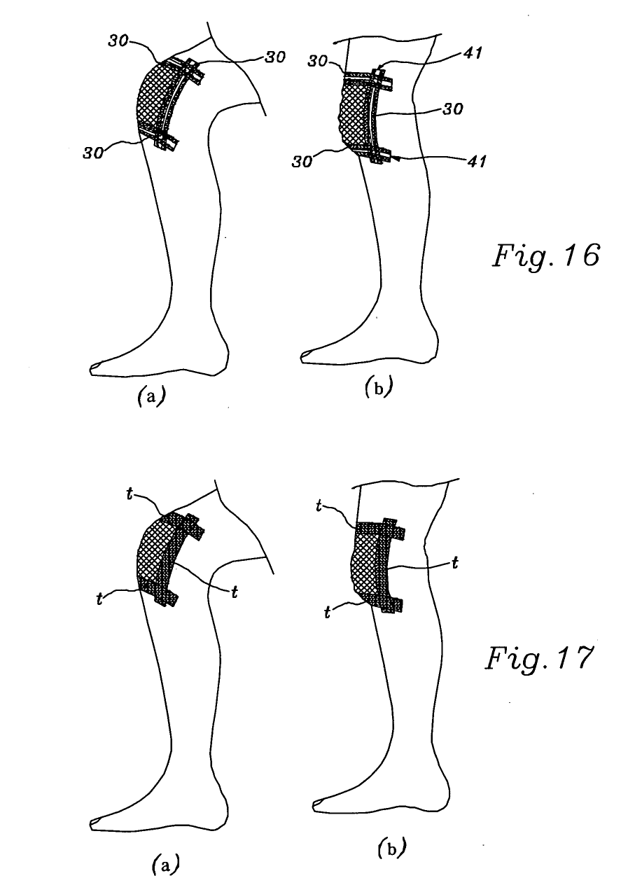

Please refer to the FIGS. 14 through 17, the close sticking way of the fixing gauze by the present invention applied on the wound at the joints of the human elbow or knee (as shown in view-a and b of the FIG. 14 as well as view-a and b of the FIG. 16) is the same as that by the conventional medical adhesive bandage t (as shown in view-a and b of the FIG. 15 as well as view-a and b of the FIG. 17); Hence, no habit in the close sticking way will be changed.

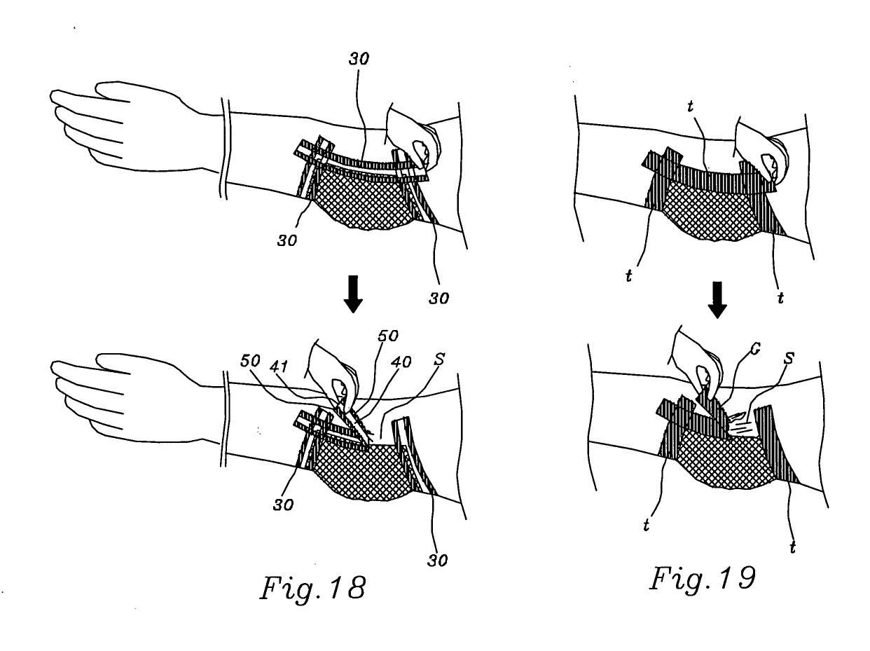

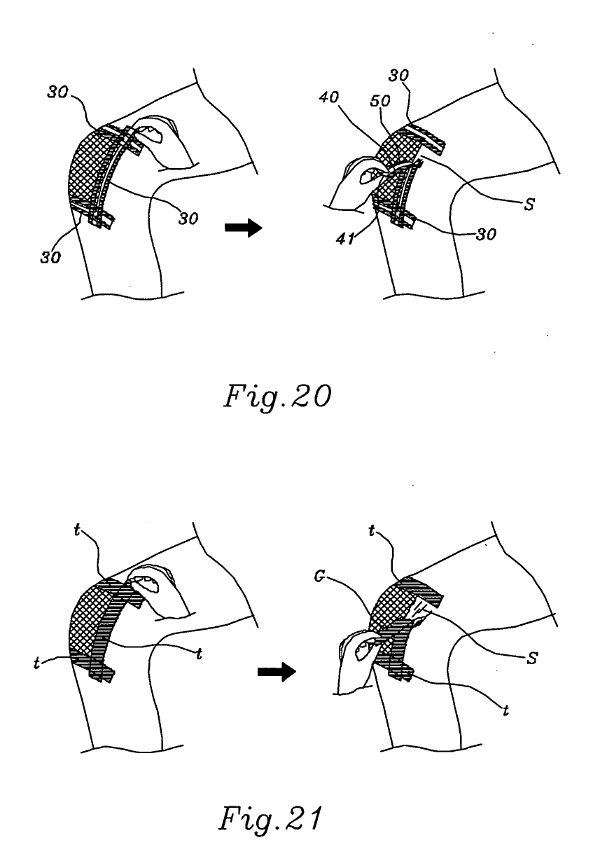

Please refer to the FIGS. 18 through 21, if said adhesive bandage is to be ripped off upon changing medical dressing, just align the fingers with either glue-less indentation 41 of both crosswise end lateral sides 33 on the strip substrate 30 as grip fulcrum for lifting up to achieve the purpose of tearing off said strip substrate 30 from the skin epidermis S (as shown in the FIGS. 18 and 20) without painful feeling by such tearing off action; Indeed, it speeds up the completely ripping off effect in addition to the simplicity and easiness in ripping off said adhesive bandage. Contrarily, for ripping off the conventional medical adhesive bandage t, a tiny edge or corner of medical adhesive bandage t is first lifted by one finger, then by means of two fingers griping such lifted tiny edge or corner as grip fulcrum for lifting up to tear off the whole medical adhesive bandage t from the skin (as shown in the FIGS. 19 and 21); The painful feeling by such tearing off procedure is serious owing to the adhesive glue completely contact and stick with the skin epidermis S.

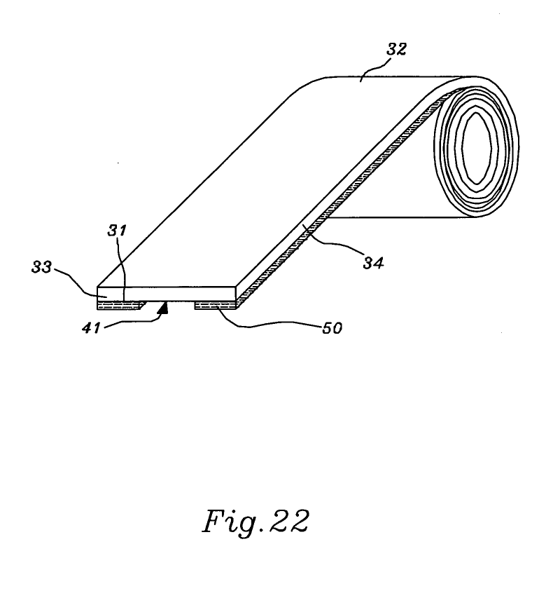

Referring to FIG. 22, the strip substrate 30 in the first preferred embodiment of present invention can further be formed into a continual flat strip for being enrolled into roll-type with a sticking front 31, a non-sticking back 32, two short end of lateral sides 33 and two lengthwise long lateral sides 34. Wherein a continual strip of non-glued area 40 is disposed between both of lengthwise long lateral sides 34 and two continual strips of glued area 50 are juxtaposed on both lengthwise sides of said non-glued area 40 respectively on said sticking front 31, so as a pair of glue-less indentations 41 are formed on both end sides of said non-glued area 40 and being flush with both short end of lateral sides 33 of said strip substrate 30 (as shown in the FIGS. 9 and 22).

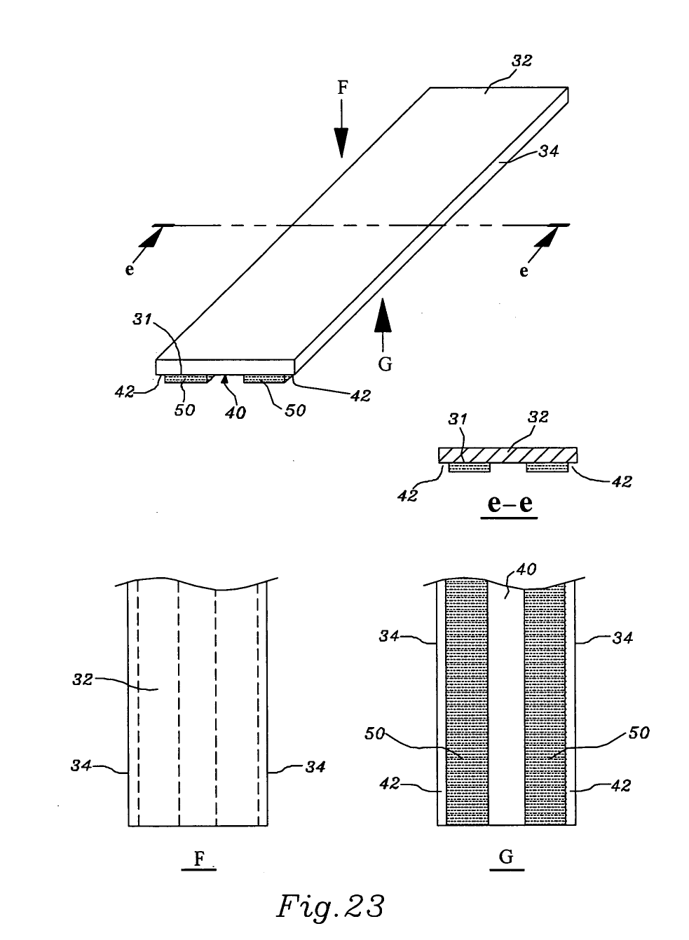

Please refer to the FIG. 23, which is the second preferred embodiment of the present invention, wherein a further pair strips of juxtaposed slimmer non-glued area 42 are inwards disposed abut against said pair of lengthwise long lateral sides 34 of said strip substrate 30 and adjacent to the pair strips of glued area 50 on the sticking front 31 of the strip substrate 30 so that said pair of lengthwise long lateral sides 34 will not become dirty due to touching and accumulating dust after the strip substrate 30 being enrolled into roll-type.

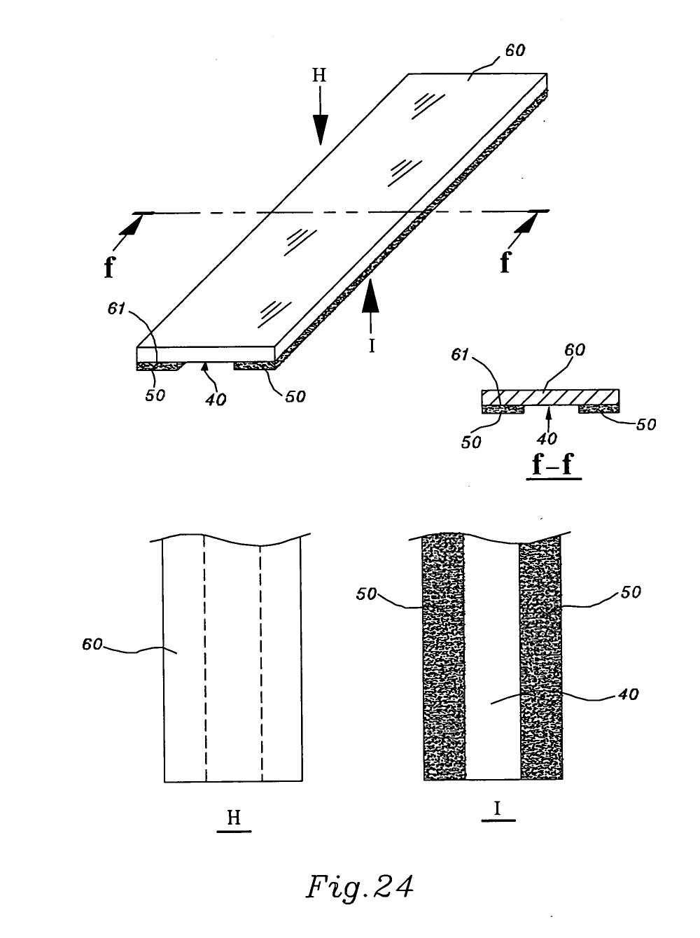

Please refer to the FIG. 24, which is the third preferred embodiment of the present invention, wherein the strip substrate 60 is further disposed into translucent or transparent flat shape and the pair strips of glued area 50 on the sticking front 61 of said strip substrate 60 are spread with color glue so that the ripping off efficiency and the management effect of induce arrangement can be increased by the variety and identification of the glue color.

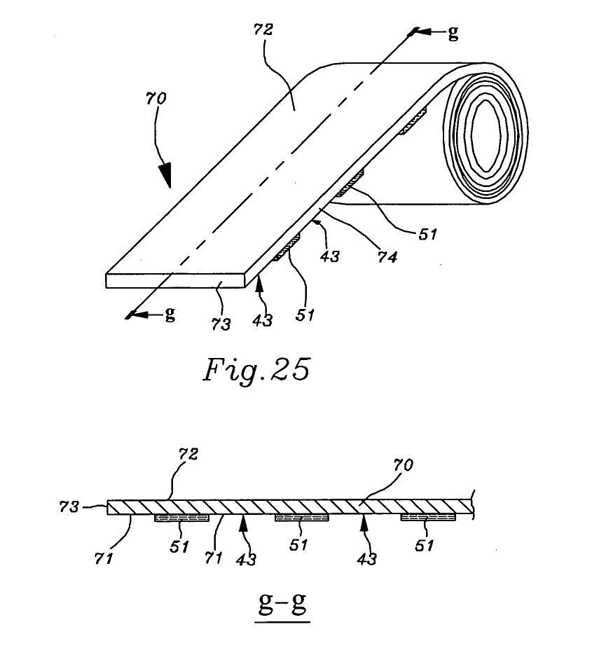

Referring to FIG. 25 illustrate a forth preferred embodiment of the present invention, in which the strip substrate 70 is further disposed into continual flat strip shape and is enrolled into roll-type comprises a sticking front 71, a non-sticking back 72, two short end sides 73 and two lengthwise long lateral sides 74. The areas of said sticking front 71 and non-sticking back 72 are greater than the two short end sides 73 and two lengthwise long lateral sides 74. Wherein a plurality of glued areas 51 whose both ends being flush with the pair of lengthwise long lateral sides 74 and are transversely disposed along the lengthways strip substrate 70 in equivalent dividing space such that some non-glued area 43are separating arranged on said sticking front 71 of said strip substrate 70.

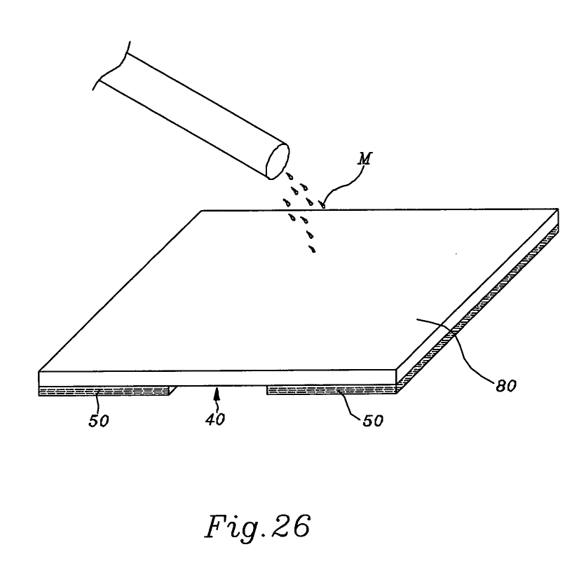

Please refer to the FIG. 26, which is the fifth preferred embodiment of the present invention, wherein the strip substrate 80 is made of nanometer material with both effect of water molecular permeability for applied medicinal liquid M required by the medical treatment, but obstructive ability keep bacteria from permeating.

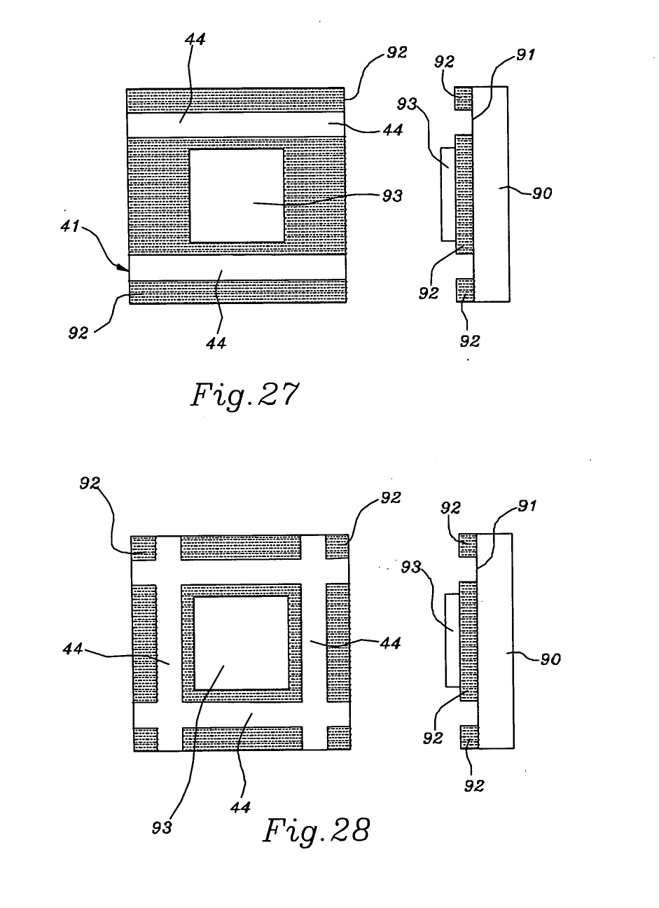

FIGS. 27 and 28 illustrate a exemplary embodiment of the present invention which is applied in the large-sized sticking plaster. A pair strips of juxtaposed non-glued area 44 are respectively disposed between one pair of sides 92 and the central dressing gauze area 93 on the sticking front 91 of the plaster substrate 90 (as shown in the FIG. 27); or two pairs of parallel juxtaposed non-glued area 44 are respectively disposed between two pair of sides 92 and the central dressing gauze area 93 on the sticking front 91 of the plaster substrate 90 (as shown in the FIG. 28). By means of glue-less indentation 41, which is formed on flush end between any strip non-glued area 44 and the corresponding sides 92, the large-sized sticking plaster can be easily and quickly ripped off from the skin epidermis S.

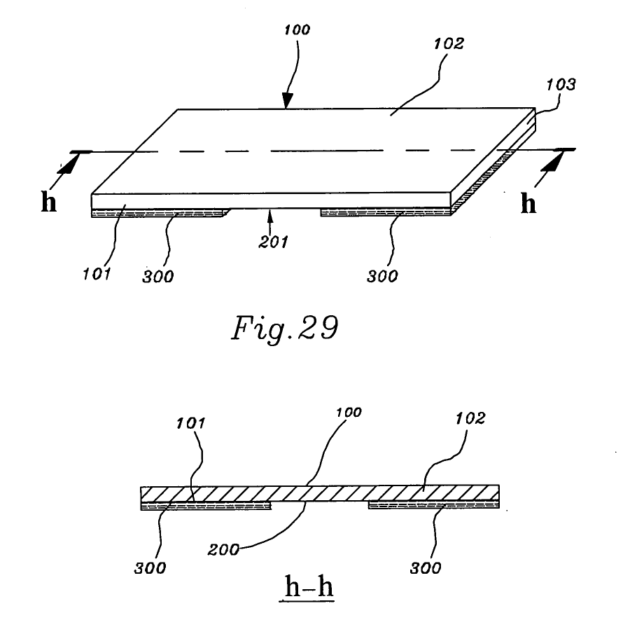

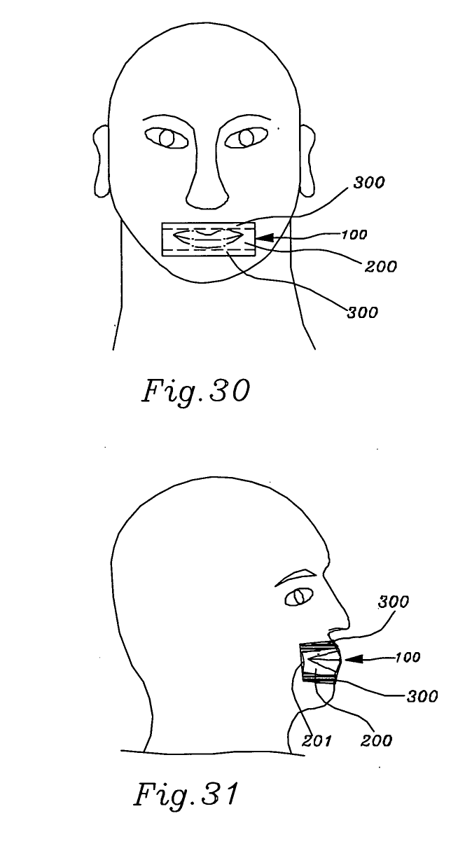

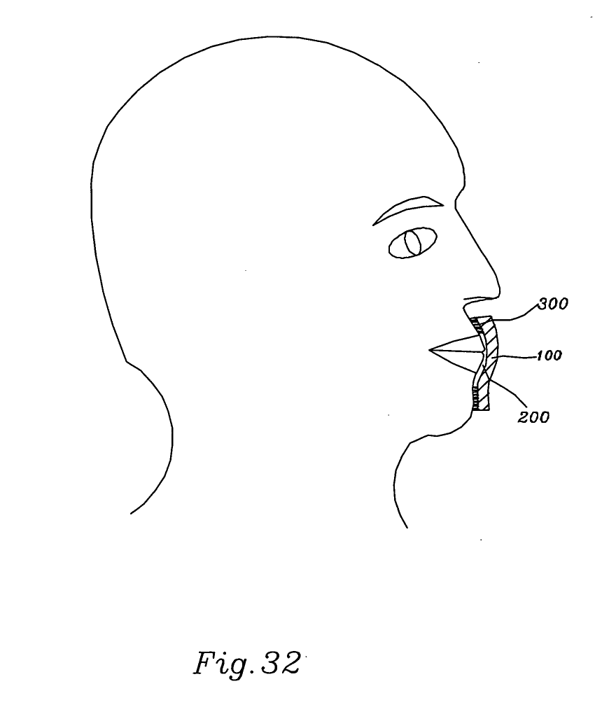

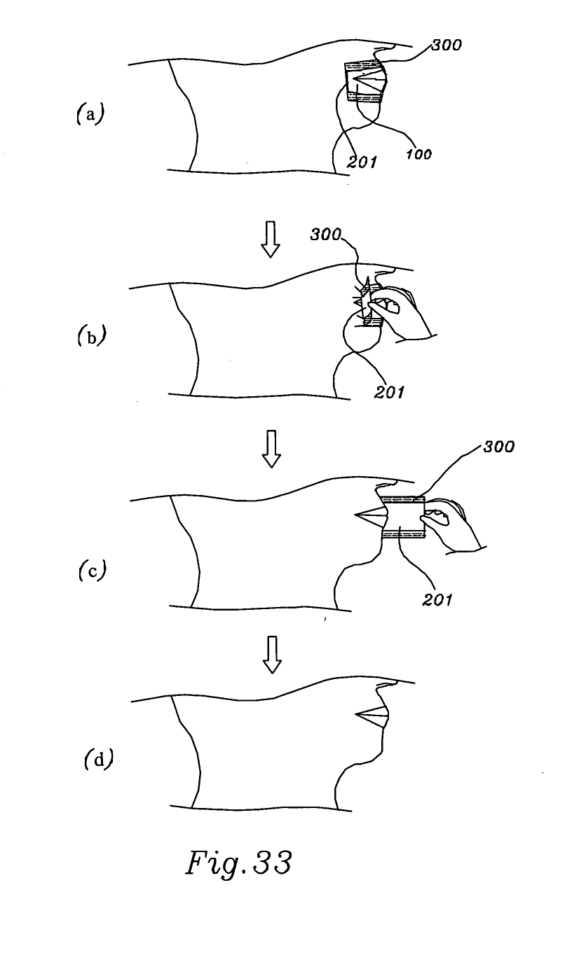

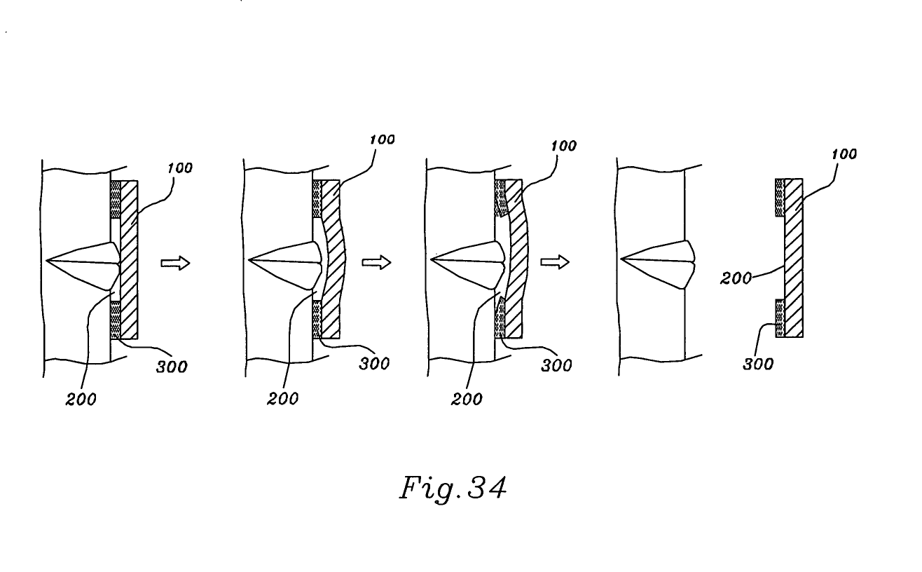

Please refer to the FIGS. 29 through 34, which is the exemplary embodiment of the present invention applied in the sticking bandage of snore-preventing. As shown in the FIG. 29, the bandage substrate 100 is has a flat strip shape comprises a sticking front 101, a non-sticking back 102 and four sides 103. The areas of said sticking front 101 and non-sticking back 102 are greater than those of four sides 103. Wherein a strip non-glued area 200 is disposed in the center of said sticking front 101 and a pair strips of parallel juxtaposed glued area 300 are disposed on both sides of said non-glued area 200 respectively, so as a pair of glue-less indentations 201 are formed on both ends of said non-glued area 200 of being not adjacent said glued area and being flush with the side 33 of the bandage substrate 100. By means of one strip said non-glued area 200 and two strips said glued area 300 on the sticking front 101 of the bandage substrate 100, the sticking bandage can closely stick on the outer skin epidermis S around the upper lip L1 and the lower lip L2 (as shown in the FIGS. 30, 31 and 32) so that closed upper lip L1 and lower lip L2 is accommodated in said non-glued area 200 without uncomfortable feeling caused by the sticking and stimulus from glue. If the sticking bandage is to be ripped off, just align the fingers with either glue-less indentation 201 of both crosswise end sides 103 on the bandage substrate 100 as grip fulcrum for lifting up to achieve the purpose of tearing off said bandage substrate 100 from the skin epidermis S (as shown in view-a, b, c, d of the FIGS. 33 and view-a, b, c, d of the FIGS. 34) without painful feeling of the upper lip L1 and the lower lip L2 being torn off by the glue sticking; Indeed, it has both effects in simplicity and easiness in quickly ripping off said adhesive bandage in addition to elimination of painful feeling in tearing off.

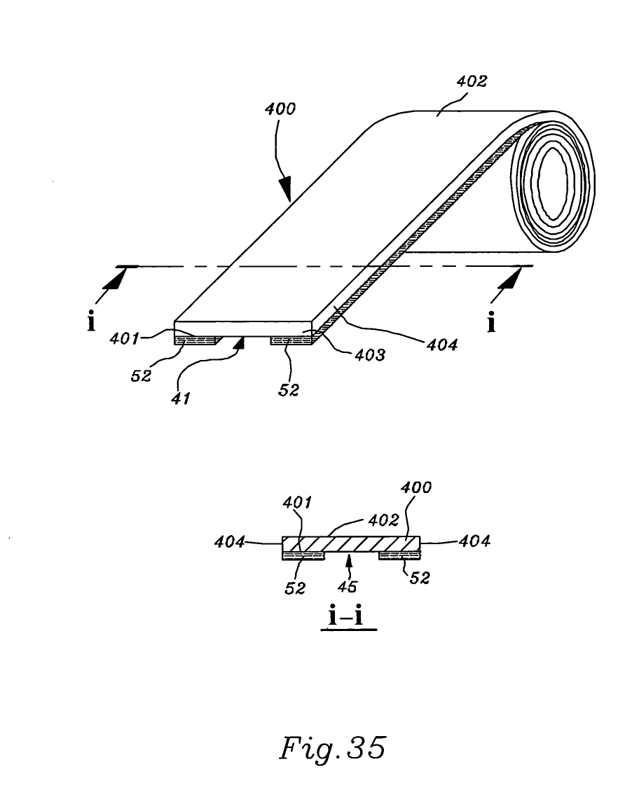

Please refer to the FIG. 35, the present invention further applied in the simple and easy tearing-off adhesive tape used in the article. The strip substrate 400 is continual flat strip shape for being enrolled into roll-type and has a sticking front 401, a non-sticking back 402, two short end sides 403 and two lengthwise long lateral sides 404. And, both areas of said sticking front 401 and non-sticking back 402 are greater than those of two short end sides 403 and two lengthwise long lateral sides 404. The feature is: a continual strip of non-glued area 45 is disposed between both of lengthwise long lateral sides 404 and two continual strips of glued area 52 are juxtaposed on both lengthwise sides of said non-glued area 45 respectively on the sticking front 401, wherein a pair of glue-less indentations 41 are formed on both strip substrate 400 and being flush with said short end sides 403.

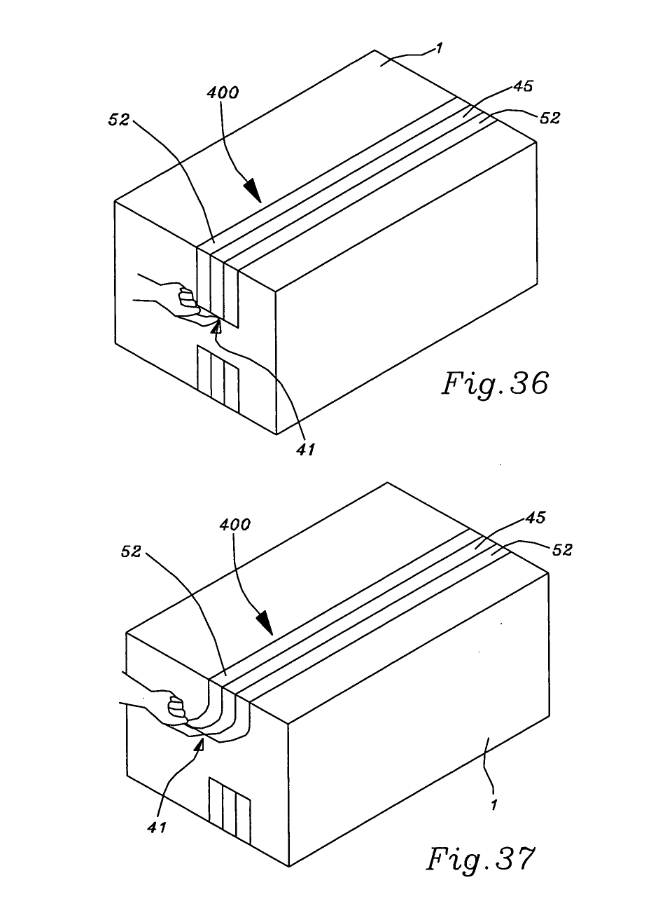

Please refer to the FIGS. 36 and 37, when the strip substrate 400 is applied to close stick and seal the box body 1, the operation way is same as that of conventional adhesive tape. If the adhesive tape is to be ripped off, just align the fingers with either glue-less indentation 41 of both short end sides 403 on the strip substrate 400 as grip fulcrum for lifting up to achieve the purpose of tearing off whole said adhesive tape from the box body 1.

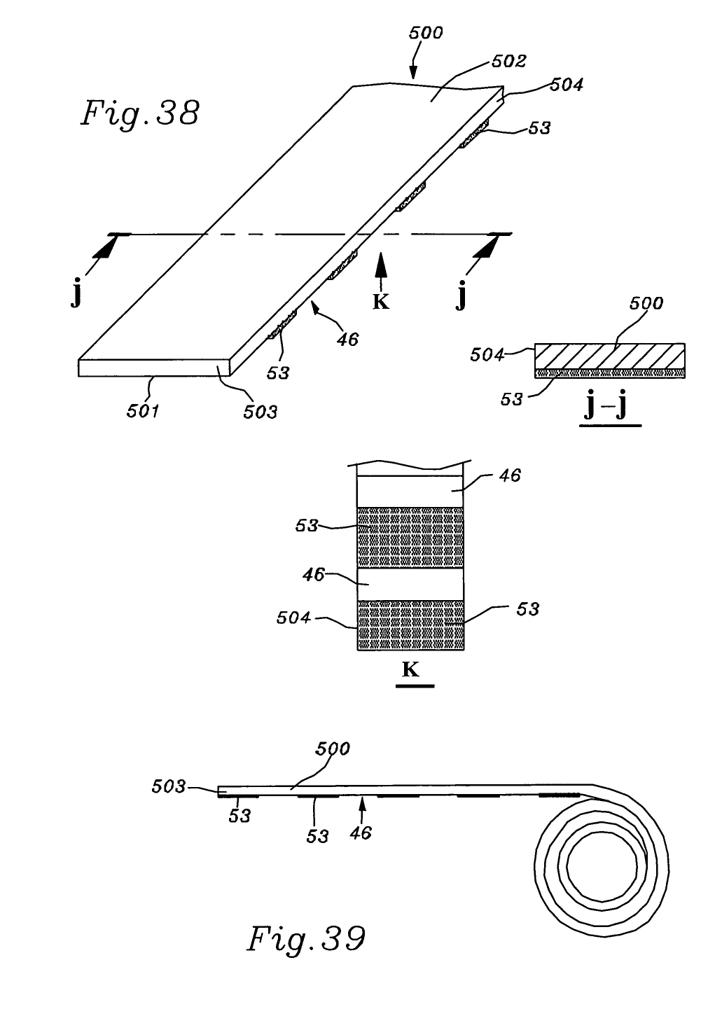

Please refer to the FIGS. 38 and 39, which is the second exemplary embodiment of the present invention applied in the simple and easy tearing-off adhesive tape used in the article. The strip substrate 500 is continual flat strip shape for being enrolled into roll-type with some glued areas 53 being disposed on the sticking front 501; Those said glued areas 53, whose both ends being flush with the pair of lengthwise long lateral sides 504, are transversely disposed along the lengthways strip substrate 500 in equivalent dividing space such that some non-glued area 46 are separating arranged on said sticking front 501 of said strip substrate 500.

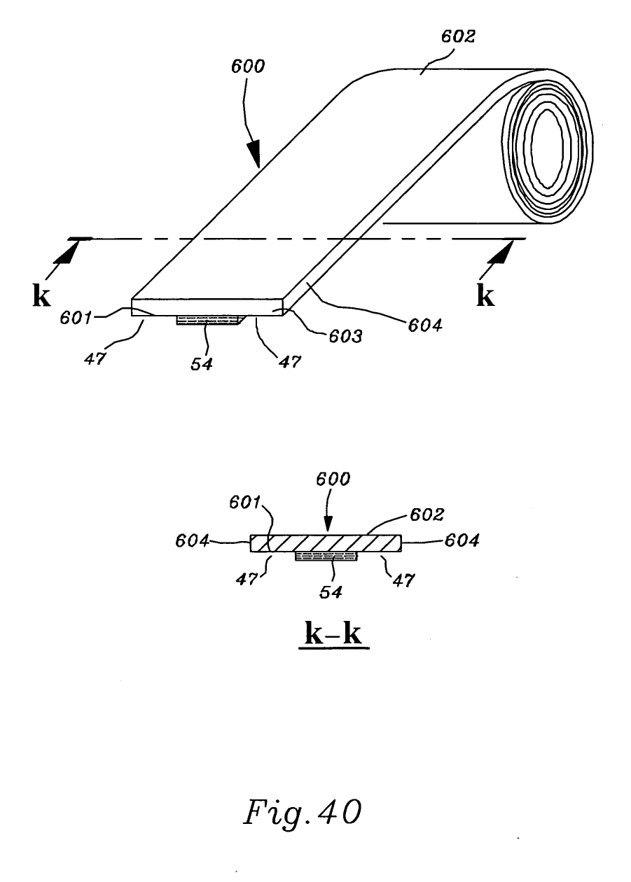

Please refer to the FIGS. 40, which is the third exemplary embodiment of the present invention applied in the simple and easy tearing-off adhesive tape used in the article. The strip substrate 600 is continual flat strip shape for being enrolled into roll-type and has a sticking front 601, a non-sticking back 602, two short end sides 603 and two lengthwise long lateral sides 604. And, both areas of said sticking front 601 and non-sticking back 602 are greater than those of two short end sides 603 and lengthwise long lateral sides 604. The feature is: a continual strip of glued area 54 is disposed between both of lengthwise long lateral sides 604 and two continual strips of non-glued area 47 are juxtaposed on both lengthwise sides of said glued area 54 and flush with two said lengthwise long lateral sides 604 respectively on the sticking front 601.

In conclusion, the present invention really has multiple application practicality and improvement in both of the medical treatment and industry, which should conform to the essential requirement of the invention patent. Hence we submit our application of the present invention in accordance with the laws and regulations.

What is claimed is:

1. An adhesive bandage with reduced pain during removal, which is a flat strip substrate comprises a sticking front, a non-sticking back and four lateral sides. The areas of said sticking front and non-sticking back are greater than the four lateral sides, wherein a strip of non-glued area is disposed between both of opposite lateral sides and two strips of glued area are juxtaposed on both sides of said non-glued area respectively on said sticking front. Therefore, a pair of glue-less indentations are formed on both end sides of said non-glued area but not adjacent said glued area of said strip substrate and being flush with both lateral sides.

2. An adhesive bandage with reduced pain during removal as recited in the claim 1, wherein said strip substrate can further be formed into a continual flat strip for being enrolled into roll-type with a sticking front, a non-sticking back, two short end of lateral sides and two lengthwise long lateral sides. Wherein a continual strip of non-glued area is disposed between both of lengthwise long lateral sides and two continual strips of glued area are juxtaposed on both lengthwise sides of said non-glued area respectively on said sticking front, so as a pair of glue-less indentations are formed on both end sides of said non-glued area and being flush with both short end of lateral sides of said strip substrate.

3. An adhesive bandage with reduced pain during removal as recited in the claim 1, wherein said strip substrate is further disposed into continual flat strip shape and is enrolled into roll-type comprises a sticking front, a non-sticking back, two short end sides and two lengthwise long lateral sides. The areas of said sticking front and non-sticking back are greater than the two short end sides and two lengthwise long lateral sides. Wherein a plurality of glued areas whose both ends being flush with the pair of lengthwise long lateral sides and are transversely disposed along the lengthways strip substrate in equivalent dividing space such that some non-glued area are separating arranged on said sticking front of said strip substrate.

4. An adhesive bandage with reduced pain during removal as recited in the claims 1, 2 and 3, wherein a further pair strips of juxtaposed slimmer non-glued area are inwards disposed abut against said pair of lengthwise long lateral sides of said strip substrate and adjacent to the pair strips of glued area on the sticking front of the strip substrate.

5. An adhesive bandage with reduced pain during removal as recited in the claims 1, 2 and 3, wherein a further strip of slimmer non-glued area is inwards disposed abut against anyone of said pair of parallel lengthwise long lateral sides of said strip substrate and adjacent to the strip of glued area on the sticking front of the strip substrate.

6. An adhesive bandage with reduced pain during removal as recited in the claims 1, 2, 3, 4, and 5, wherein said strip substrate is further disposed into translucent or transparent flat shape and the pair strips of glued area on the sticking front of said strip substrate are spread with color glue.

7. An adhesive bandage with reduced pain during removal as recited in the claims 1, 2, 3, 4, and 5, wherein said strip substrate is made of nanometer material.

8. A large-sized sticking plaster with reduced pain during removal comprises a pair strips of parallel juxtaposed non-glued area being respectively disposed between one pair of sides and the central dressing gauze area on the sticking front of the plaster substrate, wherein a pair of glue-less indentations are formed on both ends of said non-glued area of not being adjacent said central dressing gauze area and being flush with both said sides of the bandage substrate.

9. A large-sized sticking plaster with reduced pain during removal as recited in the claim 8, wherein two pair strips of parallel juxtaposed non-glued area being respectively disposed between two pair of sides and the central dressing gauze area on the sticking front of the plaster substrate, wherein two pair of glue-less indentations are formed on both ends of said non-glued area of not being adjacent said central dressing gauze area and being flush with four said sides of the bandage substrate.

10. A sticking bandage of snore-preventing with reduced pain during removal whose bandage substrate is flat strip shape of having a sticking front, a non-sticking back and four sides. And, both areas of said sticking front and non-sticking back are greater than those of four sides The feature is: a strip non-glued area is disposed in the center of a pair of opposite said sides on said sticking front and a pair strips of parallel juxtaposed glued area are disposed on both sides of said non-glued area respectively, wherein a pair of glue-less indentations are formed on both ends of said non-glued area of being not adjacent said glued area and being flush with the side of the bandage substrate.

11. A sticking bandage of snore-preventing with reduced pain during removal as recited in the claim 10, wherein said bandage substrate is flat strip shape for being enrolled into roll-type and having a sticking front, a non-sticking back and two crosswise short end sides plus two parallel lengthwise long lateral sides; And, both areas of said sticking front and non-sticking back are greater than the those of two crosswise short end sides and two parallel lengthwise long lateral sides; The feature is: a continual strip of non-glued area is disposed between both of opposite parallel lengthwise long lateral sides and two continual strips of glued area are parallel juxtaposed on both lengthwise sides of said non-glued area respectively on said sticking front, wherein a pair of glue-less indentations are formed on both crosswise end sides of said non-glued area and being flush with both crosswise short end sides of said bandage substrate.

12. A sticking bandage of snore-preventing with reduced pain during removal as recited in the claim 10, wherein said bandage substrate is flat strip shape for being enrolled into roll-type and having a sticking front, a non-sticking back and two crosswise short end sides plus two parallel lengthwise long lateral sides ; And, both areas of said sticking front and non-sticking back are greater than the those of two crosswise short end sides and two parallel lengthwise long lateral sides; The feature is: some glued areas, whose both ends being flush with the pair of parallel lengthwise long lateral sides, are transversely disposed along the lengthways bandage substrate in equivalent dividing space such that some non-glued area are separating arranged on said sticking front of said strip bandage substrate.

13. A sticking bandage of snore-preventing with reduced pain during removal as recited in the claims 10, 11 and 12, wherein an additional pair strips of parallel juxtaposed slimmer non-glued area are inwards disposed abut against said pair of parallel lateral sides of said bandage substrate and adjacent to the pair strips of glued area on the sticking front of the bandage substrate.

14. A sticking bandage of snore-preventing with reduced pain during removal as recited in the claims 10, 11 and 12, wherein an additional strip of slimmer non-glued area is inwards disposed abut against anyone of said pair of parallel lateral sides of said bandage substrate and adjacent to the strip of glued area on the sticking front of the bandage substrate.

15. A sticking bandage of snore-preventing with reduced pain during removal as recited in the claims 10, 11, 12 and 13, wherein said bandage substrate is further disposed into translucent or transparent flat shape and the pair strips of glued area on the sticking front of said bandage substrate are spread with color glue.

16. A sticking bandage of snore-preventing with reduced pain during removal as recited in the claims 10, 11, 12, 13 and 14, wherein said bandage substrate is made of nanometer material.

17. A simple and easy tearing-off adhesive tape used for the article whose strip substrate is continual flat strip shape for being enrolled into roll-type and has a sticking front, a non-sticking back and two crosswise short end sides plus two parallel lengthwise long lateral sides And, both areas of said sticking front and non-sticking back are greater than those of four short end sides and long lateral sides ; The feature is: a continual strip of non-glued area is disposed between both of opposite parallel lengthwise long lateral sides and two continual strips of glued area are parallel juxtaposed on both lengthwise sides of said non-glued area respectively on the sticking front, wherein a pair of glue-less indentations are formed on both crosswise strip substrate and being flush with said short end sides.

18. A simple and easy tearing-off adhesive tape used for the article as recited in the claim 17, wherein said strip substrate is flat strip shape for being enrolled into roll-type of having a sticking front, a non-sticking back and two crosswise short end sides plus two parallel lengthwise long lateral sides; And, both areas of said sticking front and non-sticking back are greater than the those of two crosswise short end sides and two parallel lengthwise long lateral sides ; The feature is: some glued areas, whose both ends being flush with the pair of parallel lengthwise long lateral sides, are transversely disposed along the lengthways strip substrate in equivalent dividing space such that some non-glued area are separating arranged on said sticking front of said strip substrate.

19. A simple and easy tearing-off adhesive tape used for the article as recited in the claims 17 and 18, wherein an additional pair strips of parallel juxtaposed slimmer non-glued area are inwards disposed abut against said pair of parallel lateral sides of said strip substrate and adjacent to the pair strips of glued area on the sticking front of the strip substrate.

20. A simple and easy tearing-off adhesive tape used for the article as recited in the claims 17 and 18, wherein an additional strip of slimmer non-glued area is inwards disposed abut against anyone of said pair of parallel lateral sides of said strip substrate and adjacent to the strip of glued area on the sticking front of the strip substrate.

21. A simple and easy tearing-off adhesive tape used for the article as recited in the claims 17, 18, 19 and 20, wherein said strip substrate is further disposed into translucent or transparent flat shape and the pair strips of glued area on the sticking front of said strip substrate are spread with color glue.

22. A simple and easy tearing-off adhesive tape used for the article as recited in the claim 17, wherein said strip substrate is flat strip shape for being enrolled into roll-type of having a sticking front, a non-sticking back and two crosswise short end sides plus two parallel lengthwise long lateral sides; And, both areas of said sticking front and non-sticking back are greater than the those of two crosswise short end sides and two parallel lengthwise long lateral sides; The feature is: a continual strip of glued area is disposed between both of opposite parallel lengthwise long lateral sides and two continual strips of non-glued area are parallel juxtaposed on both lengthwise sides of said glued area and flush with two said long lateral sides respectively on the sticking front.

23. A simple and easy tearing-off adhesive tape used for the article as recited in the claim 22, wherein a continual strip of glued area is disposed between both of opposite parallel lengthwise long lateral sides and one continual strip of non-glued area is disposed on anyone of both lengthwise sides of said glued area and flush with said long lateral side on the sticking front.
